(19) United States
(12) Patent Application Publication
(10) Pub. No.: US 2025/0114723 A1
(43) Pub. Date: Apr. 10, 2025

(54) A SYSTEM AND METHOD FOR DISTILLATION

(71) Applicant: PRAJ INDUSTRIES LIMITED, Pune (IN)

(72) Inventors: Ghansham Baburao DESHPANDE, Pune (IN); Ajit Prabhakar DESHMUKH, Pune (IN)

(21) Appl. No.: 18/276,718

(22) PCT Filed: Jun. 1, 2023
(86) PCT No.: PCT/IN2023/050511

§ 371 Date: Aug. 10, 2023

(30) Foreign Application Priority Data
Jul. 1, 2022 (IN) 202221038264

(51) Int. Cl. B01D 3/14; B01D 1/28; B01D 3/00; B01D 5/00; B01D 53/14; B01D 53/26

(52) U.S. Cl. CPC B01D 3/148; B01D 1/2818; B01D 3/002; B01D 5/006; B01D 53/14; B01D 53/265; B01D 2257/80

(57) ABSTRACT

The present invention relates to a system and method for distillation to reduce steam consumption has been disclosed. The system comprises of an analyser column 11, multiple pressure booster units (fan set-I 79, fan set-II 24, and fan set-III 29), a rectifier column 15, a plurality of evaporator units (30,12), a splitter unit 05, a plurality of de-superheating units (03, 80), and additional DDGS dryer unit 25. The integration of pressure booster units (fan set-I 79, fan set-II 24, and fan set-III 29) and additional DDGS dryer unit 05 increases the steam (vapor) production and reduces the steam (vapor) consumption in the system from external source and balances the optimization of process energy requirements, energy cost, and process carbon intensity.

### CROSS-REFERENCE TO RELATED APPLICATIONS AND PRIORITY

The present application claims priority from Indian patent application number (202221038264) filed on (Jan. 7, 2022), incorporated herein by a reference.

### FIELD OF THE INVENTION

The present invention, in general, relates to an ethanol distillation system, and more particularly, relates to an improved ethanol distillation system having multiple sets of pressure booster units.

### BACKGROUND OF THE INVENTION

Distillation or classical distillation enables the separation of two or more components or substances from a liquid mixture by using selective boiling and condensation temperatures. Further, the process may also be applied to separate two liquids having a different boiling point.

The distillation process may further be used for obtaining ethanol. The present ethanol distillation system use steam as an energy driver, wherein the steam is generated by burning natural gas/Coal/Fossil Fuel. However, the usage of such carbon-based fuel steam generation increases the greenhouse gas emission value, and the reduction of the energy requirement decreases the CI (Carbon Intensity) number for the production of ethanol.

Thus, there exists a long-felt need to design and develop an improved ethanol distillation system that does not rely on carbon-based fuel for steam generation.

### SUMMARY OF THE INVENTION

Before the present disclosure for an improved system and method for ethanol distillation for reduction of steam is described, it is to be understood that this application is not limited to the particular systems, and methodologies described, as there can be multiple possible embodiments which are not expressly illustrated in the present disclosure. It is also to be understood that the terminology used in the description is for the purpose of describing the particular versions or embodiments only and is not intended to limit the scope of the present application. This summary is not intended to identify essential features of the claimed subject matter nor is it intended for use in determining or limiting the scope of the claimed subject matter

In an implementation of the present disclosure, a method for distillation has been disclosed. The method may include transferring of a preheated feed stream to an analyser column for stripping vapor from the preheated feed stream. Further, compressing the stripped vapor in a fan set-I, in one or more stages to form compressed vapor. Further. transferring the compressed vapor from the fan set-I to a rectifier column and rectifying the compressed vapor in the rectifier column to obtain the rectified vapor Further, transferring the rectified vapor from the rectifier column to an evaporator unit and condensing the rectified vapor to form rectified condensate in the evaporator unit. Further, transferring the rectified condensate to a dehydration section for further processing to obtain ethanol. The water vapor formed during the condensation process in the evaporator unit may be transferred to further fan set units.

In an implementation of the present disclosure, a system for distillation has been disclosed. The system as disclosed may comprise a preheater, an analyser column, a fan set-I, a rectifier column, and an evaporator unit. The analyser column may be configured to receive a preheated feed stream from the preheater, and may be configured to strip-off the vapor from the received preheated feed stream. Further the fan set-I may be configured to receive the stripped vapor from the analyser column and may be configured to compress the stripped vapor in one or more stages to form a compressed vapor. Further, the rectifier column may be configured to receive the compressed vapor from the fan set-I and may be configured to rectify the compressed vapor to form a rectified vapor. The evaporator unit may be configured to receive the rectified vapor from the rectifier column and may be configured to condense the rectified vapor to form a rectified condensate. Further, the evaporator unit may be configured to transfer the rectified condensate to a dehydration section for further concentration of ethanol from the rectified condensate. The water vapor formed during the condensation process in the evaporator unit may be transferred to further fan set units.

### BRIEF DESCRIPTION OF DRAWINGS

The detailed description is described with reference to the accompanying figures. The same numbers are used throughout the drawings to refer like features and components.

FIG. 1. illustrates a system and method for distillation, in accordance with an exemplary embodiment.

FIG. 2. illustrates a liquefication and a dehydration section associated with a system and method for distillation, in accordance with an exemplary embodiment.

FIG. 3. illustrates a DDGS dryer system associated with a system and method for distillation. in accordance with an exemplary embodiment.

### DETAILED DESCRIPTION OF THE INVENTION

Reference throughout the specification to “various embodiments,” “some embodiments,” “one embodiment,” or “an embodiment” means that a particular feature, structure, or characteristic described in connection with the embodiment is included in at least one embodiment. Thus, appearances of the phrases “in various embodiments,” “in some embodiments,” “in one embodiment,” or “in an embodiment” in places throughout the specification are not necessarily all referring to the same embodiment. Furthermore, the particular features, structures or characteristics may be combined in any suitable manner in one or more embodiments. Following is an example which is illustrative only and invention accommodates any and every variation of the example provided below that shall serve the same purpose and is obvious to a person skilled in the art.

The present subject matter relates to a system and method for distillation for reduced steam consumption. The system comprises a preheater, an analyser column, a rectifier column, a first and second evaporator units, a first and second de-superheating units, multiple pressure booster units (Fan Set I, II, and III) and an exhaust column. The Fan Set units may comprise a multiple set of fans. The Fan Set units may be placed at multiple locations for pressurizing the vapors and substituting it with fuel-based steam during the distillation cycle. Further, the method may include preheating a fermented wash in the preheater and feeding a preheated feed stream to the analyser column. The analyser column may perform stripping of vapor from the preheated feed stream. Further, the stripped vapor may be fed to the Fan set I. The Fan set I may be configured to pressurise the stripped vapor to obtain compressed vapor in one or more stages. Further, the compressed vapor may be fed to the rectifier column. The compressed vapor may increase the operating pressure and temperature of the rectifier column, and thereby increases throughput of the rectifier column. Also, rectifier may be configured to receive feed from a dehydration section for recovery of ethanol. The rectifier column may be configured to rectify the compressed vapor to obtain rectified vapor. Further, the rectified vapor from rectifier column may be fed to the first evaporator unit for condensation. The first evaporator unit may be configured to fed part of condense vapor to the rectifier as reflux and left over condense vapor to the dehydration section for further concentration of ethanol.

Further, the water vapor from the first evaporator may be fed to the Fan set II. The Fan set II may be configured to pressurise the water vapor to superheated water vapor in one or more stages. The superheated water vapor may be fed to the first de-superheater unit to obtain saturated water vapor. Further the saturated water vapor may be fed to the splitter unit. The splitter unit may be configured to split the saturated water vapor into first and second saturated water vapor streams. Further, the second saturated water vapor stream may be fed to the second evaporator unit. The second evaporator unit may also receive additional saturated water vapor stream from the dehydration section along with second saturated water vapor stream from the splitter unit. Further, the second saturated water vapor stream and additional saturated water vapor stream may be fed to the analyser column to act as a heat source. Further, the first saturated water vapor stream from the splitter unit may be received by the Fan set III. The fan set III may be configured to compress the first saturated water vapor stream in one or more stages to obtain superheated water vapor. Further, the second de-superheater unit may be configured to receive and de-superheat the superheated water vapor to obtain saturated water vapor. The saturated water vapor may be further received by the exhaust column to act as a heat source.

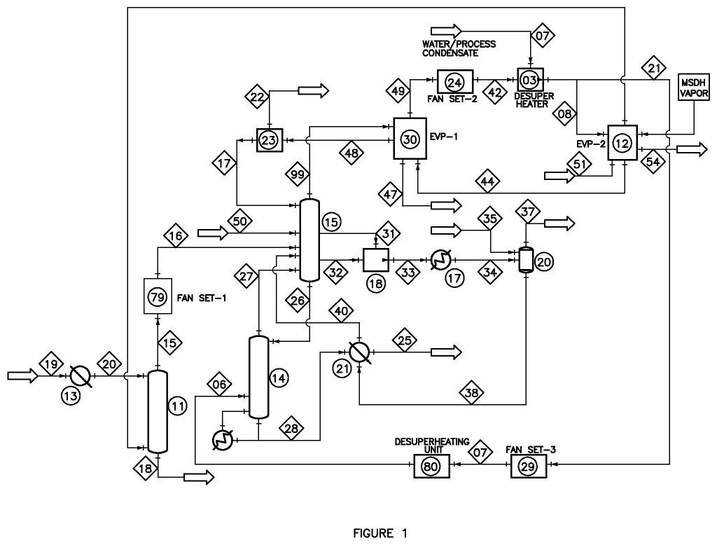

Referring to FIG. 1 illustrates a system and method for distillation for reduction of Steam, in accordance with an exemplary embodiment. The system 100 may comprise a preheater 13. The preheater may further be connected to an analyser column 11. The analyser column 11 may be further connected to a first pressure booster (Fan Set I unit), wherein the Fan Set I unit may comprise a first set of fans 79. The first set of fans 79 may be configured to increase pressure in one or more stages in series. Further, the first set of fans 79 may be connected to a rectifier column 15. Further, the rectifier column 15 may be connected to a first evaporator unit 30. Further, the first evaporator unit 30 may be connected to a second pressure booster unit (Fan Set II unit), the Fan Set II unit may comprise a second set of fans 24. The second set of fans 24 may be configured to increase pressure in one or more stages in series. The second set of fans 24 may be further connected to a first de-superheating unit 03. Further, the first de-superheating unit 03 may be connected to a splitter unit 05, the splitter unit may be further connected to a second evaporator unit 12 and a third pressure booster (Fan Set III unit), the Fan Set III unit may comprise a third set of fans 29. The second evaporator unit 12 may be further connected to the analyser column 11 and the third set of fans 29 may be connected to a second de-superheating unit 80. Further, the second de-superheating unit 80 may be connected to an exhaust column 14

Further, in accordance with the exemplary embodiment, a fermented wash 19 containing 8-20% v/v ethanol concentration may be preheated at 60-70 degree temperature in the feed preheater 13 to obtain a preheated feed stream 20. The preheated feed stream 20 may be fed to the analyser column 11 for stripping of ethanol and water stream of the preheated feed stream 20 to obtain stripped vapor 15′ from the top of the analyser column 11 operating at a pressure of 0.4 to 0.6 bar and at a temperature of 72-75 degree Centigrade The stripped vapor 15′ at top of the analyser column 11 may contain 45-65% v/v of ethanol and 30-55% of water. Further, the stripped vapor 15′ may be compressed by the Fan Set-I 79 in one or more stages to obtain compressed vapor 16 at a pressure of 1.2-1.3 bar. The obtained compressed vapor 16 may comprise ethanol and water stream at pressure 1.2-1.3 bar and flow rate of 33305 kg/h.

Further, the compressed vapor 16 at a pressure of 1.2-1.3 bar, at a temperature of 140-160 degree, and at a flow rate of 33305 kg/h may be transferred to the rectifier column 15 by the fan set-1 79 for the further rectification process. The transfer of high pressure and high temperature compressed vapor 16 to the rectifier column 15 may increase operating pressure and of rectifier column 15 and thereby may result in higher throughput to obtain rectified vapor 99. The rectified vapor 99 having temperature between 80-90 degree. pressure between 1.10-1.20 bar, and flowrate of 71710 kg/h present at the top of the rectifier column 15 may be further transferred to the first evaporator unit 30 for condensation process. The rectified vapor 99 may be condensed to form rectified condensate 48 in the first evaporator unit 30.

The part of rectified condensate 48 from the first evaporator unit 30 may be transferred back to the rectifier column 15 as a reflux 17 and the remaining rectified condensate 48 may be transferred to dehydration section 89 for further concentration of ethanol from the rectified condensate 48. After condensation process the first evaporator unit 30 may comprise of water vapor 49 at temperature between 65-75-degree, pressure 0.2-0.4 Bar, and flowrate of 27520 kg/h. The water vapors 49 present at the top of the first evaporator unit 30 may be transferred to the Fan Set-II 24. The Fan Set-II 24 may be configured to compress the water vapor 49 from 0.3 bar to 0.9 bar to obtain superheated water vapor 42 in one or more stages.

The superheated water vapors 42 may be received by the first de-superheating unit 03. The first de-superheating unit 03 may be configured to de-superheat the superheated water vapor 42 to obtain saturated water vapor 53. Further, the saturated water vapor 53 may be received by the splitter unit 05. The splitter unit 05 may be configured to split the saturated water vapor 53 into saturated first water vapor stream 21 and saturated second water vapor stream 08. Further the saturated second water vapor stream 08 at the pressure between 0.8-0.10 bar, temperature 96-99 degree, and flow rate of 20100 kg/h may be transferred to the second evaporator unit 12 by the splitter unit 05. Further, the second evaporator unit 12 may be configured to receive an additional saturated water vapor stream 90 from the dehydration section 89. The additionally received saturated water vapor stream 90 from the dehydration section 89 and saturated second water vapor stream 08 received from the splitter unit 05 may combine to form combined saturated water vapor 43. The second evaporator unit 12 may be configured to transfer the combined saturated water vapor 43 at a pressure of 0.4-0.6 bar, temperature of 80-90 degree, and flow rate of 24300 kg/h back to the analyser column 11 to make combined saturated water vapor 43 may act as a heat source.

Further the splitter unit 05 may be configured to transfer the saturated first water vapor stream 21 at a pressure of 0.8-0.10 bar and flow rate of 9800 kg/h to the fan set-III 29. The fan set-III 29 may be configured to compress the saturated first water vapor stream 21 to a pressure of 2.1 bar to form superheated water vapor 07 at flow rate of 9821 kg/h. Further, the superheated water vapor 07 may be transferred to second second de-superheating unit 80. The second second de-superheating unit 80 may be configured to de-superheat the superheated water vapor 07 to obtain saturated water vapor 06 at pressure of 2.1 bar, temperature of 127° C. and flowrate of 10471 kg/h. The saturated water vapor 06 at flow rate of 6800 kg/h may be received by the exhaust column 14 to make saturated water vapor 06 may act as a heat source.

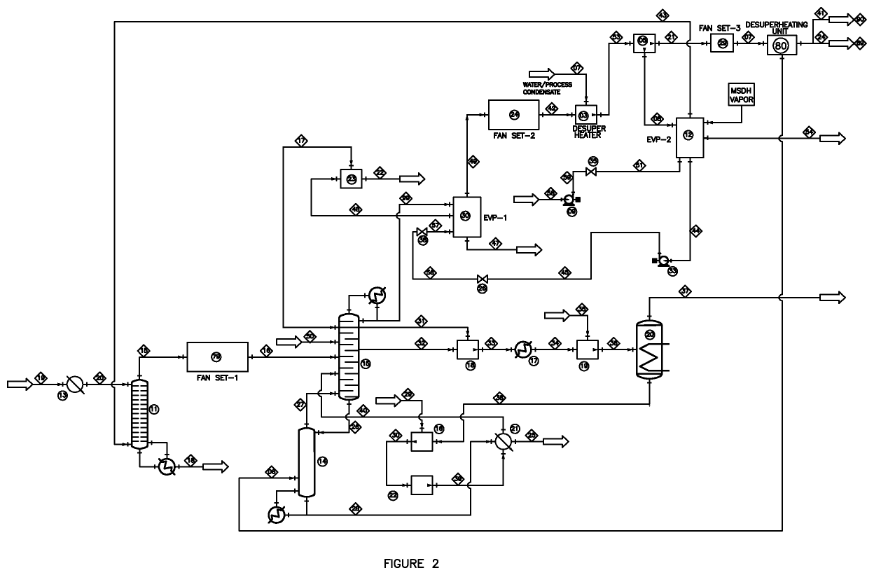

Referring to FIGS. 2 illustrates a system and method for distillation for reduction of steam, in accordance with an exemplary embodiment. The system 100 may comprise a preheater 13. The preheater may further be connected to an analyser column 11. The analyser column 11 may be further connected to a first pressure booster (Fan Set I unit), wherein the Fan Set I unit may comprise a first set of fans 79. The first set of fans 79 may be configured to increase pressure in one or more stages in series. Further, the first set of fans 79 may be connected to a rectifier column 15. Further, the rectifier column 15 may be connected to a first evaporator unit 30. Further, the first evaporator unit 30 may be connected to a second pressure booster unit (Fan Set II unit), the Fan Set II unit may comprise a second set of fans 24. The second set of fans 24 may be configured to increase pressure in one or more stages in series. The second set of fans 24 may be further connected to a first de-superheating unit 03. Further, the first de-superheating unit 03 may be connected to a splitter unit 05, the splitter unit may be further connected to a second evaporator unit 12 and a third pressure booster (Fan Set III unit), the Fan Set III unit may comprise a third set of fans 29. The second evaporator unit 12 may be further connected to the analyser column 11 and the third set of fans 29 may be connected to a second de-superheating unit 80. Further, the second de-superheating unit 80 may be connected to an exhaust column 14, a liquefaction section 90, and a dehydration section 89.

Further, in accordance with the exemplary embodiment, a fermented wash 19 containing 8-20% v/v ethanol concentration may be preheated at 60-70 degree temperature in the feed preheater 13 to obtain a preheated feed stream 20. The preheated feed stream 20 may be fed to the analyser column 11 for stripping of ethanol and water stream of the preheated feed stream 20 to obtain stripped vapor 15′ from the top of the analyser column 11 operating at a pressure of 0.4 to 0.6 bar and at a temperature of 72-75 degree Centigrade. The stripped vapor 15′ at top of the analyser column 11 may contain 45-65% v/v of ethanol and 30-55% of water. Further, the stripped vapor 15′ may be compressed by the Fan Set-I 79 in one or more stages to obtain compressed vapor 16 at a pressure of 1.2-1.3 bar. The obtained compressed vapor 16 may comprise ethanol and water stream at pressure 1.2-1.3 bar and flow rate of 33305 kg/h.

Further, the compressed vapor 16 at a pressure of 1.2-1.3 bar, at a temperature of 140-160 degree, and at a flow rate of 33305 kg/h may be transferred to the rectifier column 15 by the fan set-I 79 for the further rectification process. The transfer of high pressure and high temperature compressed vapor 16 to the rectifier column 15 may increase operating pressure and of rectifier column 15 and thereby may result in higher throughput to obtain rectified vapor 99. The rectified vapor 99 having temperature between 80-90 degree, pressure between 1.10-1.20 bar, and flowrate of 71710 kg/h present at the top of the rectifier column 15 may be further transferred to the first evaporator unit 30 for condensation process. The rectified vapor 99 may be condensed to form rectified condensate 48 in the first evaporator unit 30.

The part of rectified condensate 48 from the first evaporator unit 30 may be transferred back to the rectifier column 15 as a reflux 17 and the remaining rectified condensate 48 may be transferred to the dehydration section 89 for further concentration of ethanol from the rectified condensate 48. After condensation process the first evaporator unit 30 may comprise of water vapor 49 at temperature between 65-75-degree, pressure 0.2-0.4 Bar, and flowrate of 27520 kg/h. The water vapors 49 present at the top of the first evaporator unit 30 may be transferred to the Fan Set-II 24. The Fan Set-II 24 may be configured to compress the water vapor 49 from 0.3 bar to 0.9 bar to obtain superheated water vapor 42 in one or more stages.

The superheated water vapor 42 may be received by the first de-superheating unit 03. The first de-superheating unit 03 may be configured to de-superheat the superheated water vapor 42 to obtain saturated water vapor 53. Further, the saturated water vapor 53 may be received by the splitter unit 05. The splitter unit 05 may be configured to split the saturated water vapor 53 into saturated first water vapor stream 21 and saturated second water vapor stream 08. Further the saturated second water vapor stream 08 at the pressure between 0.8-0.10 bar, temperature 96-99 degree, and flow rate of 20100 kg/h may be transferred to the second evaporator unit 12 by the splitter unit 05. Further, the second evaporator unit 12 may be configured to receive an additional saturated water vapor stream 90 from the dehydration section 89. The additionally received saturated water vapor stream 90 from the dehydration section 89 and saturated second water vapor stream 08 received from splitter unit 05 may combine to form combined saturated water vapor 43. The second evaporator unit 12 may be configured to transfer the combined saturated water vapor 43 at a pressure of 0.4-0.6 bar, temperature of 80-90 degree, and flow rate of 24300 kg/h back to the analyser column 11 to make combined saturated water vapor 43 may act as a heat source.

Further the splitter unit 05 may be configured to transfer the saturated first water vapor stream 21 at a pressure of 0.8-0.10 bar and flow rate of 9800 kg/h to the fan set-III 29. The fan set-III 29 may be configured to compress the saturated first water vapor stream 21 to a pressure of 2.1 bar to form superheated water vapor 07 at flow rate of 9821 kg/h. Further, the superheated water vapor 07 may be transferred to second second de-superheating unit 80. The second second de-superheating unit 80 may be configured to de-superheat the superheated water vapor 07 to obtain saturated water vapor 06 at pressure of 2.1 bar, temperature of 127° C. and flowrate of 10471 kg/h, a saturated water vapor 41 for the liquefaction section 90 at flowrate of 3671 kg/h, and a saturated water vapor 24 for the dehydration section 89. The saturated water vapor 06 at flow rate of 6800 kg/h may be received by the exhaust column 14, the saturated water vapor 41 may be received by the liquefaction section 90, and the saturated water vapor 24 may be received by the dehydration section 89 to act as a heat source.

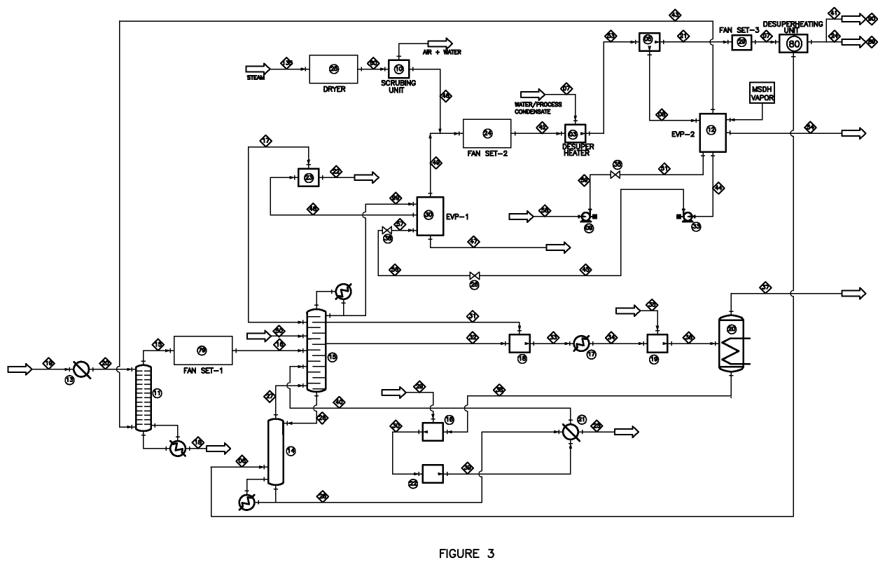

Referring to FIGS. 3 illustrates a DDGS dryer system associated with the system and method for distillation for reduction of steam, in accordance with an exemplary embodiment. The system 100 may comprise a preheater 13. The preheater may further be connected to an analyser column 11. The analyser column 11 may be further connected to a first pressure booster (Fan Set I unit), the Fan Set I unit may comprise a first set of fans 79. The first set of fans 79 may be configured to increase pressure in one or more stages in series. Further, the first set of fans 79 may be connected to a rectifier column 15. Further, the rectifier column 15 may be connected to a first evaporator unit 30. The first evaporator unit 30 may be connected to a second pressure booster unit (Fan Set II unit), the Fan Set II unit may comprise a second set of fans 24. The second set of fans 24 may be configured to increase pressure in one or more stages in series. Further an additional DDGS dryer unit 25 may also be connected to the second set of fans 24 through a scrubbing unit 10. The second set of fans 24 may be further connected to a first de-superheating unit 03. Further, the first de-superheating unit 03 may be connected to a splitter unit 05, the splitter unit may be further connected to a second evaporator unit 12 and a third pressure booster (Fan Set III unit), the Fan Set III unit may comprise a third set of fans 29. The second evaporator unit 12 may be further connected to the analyser column 11 and the third set of fans 29 may be connected to a second de-superheating unit 80. Further, the second de-superheating unit 80 may be connected to an exhaust column 14, a liquefaction section 90, and a dehydration section 89.

Further, in accordance with the exemplary embodiment, a fermented wash 19 containing 8-20% v/v ethanol concentration may be preheated at 60-70 degree temperature in the feed preheater 13 to obtain a preheated feed stream 20. The preheated feed stream 20 may be fed to the analyser column 11 for stripping of ethanol and water stream of the preheated feed stream 20 to obtain stripped vapor 15′ from the top of the analyser column 11 operating at a pressure of 0.4 to 0.6 bar and at a temperature of 72-75 degree Centigrade. The stripped vapor 15′ at top of the analyser column 11 may contain 45-65% v/v of ethanol and 30-55% of water. Further, the stripped vapor 15′ may be compressed by the Fan Set-I 79 in one or more stages to obtain compressed vapor 16 at a pressure of 1.2-1.3 bar. The obtained compressed vapor 16 may comprise ethanol and water stream at pressure 1.2-1.3 bar and flow rate of 33305 kg/h.

Further, the compressed vapor 16 at a pressure of 1.2-1.3 bar, at a temperature of 140-160 degree, and at a flow rate of 33305 kg/h may be transferred to the rectifier column 15 by the fan set-I 79 for the further rectification process. The transfer of high pressure and high temperature compressed vapor 16 to the rectifier column 15 may increase operating pressure and of rectifier column 15 and thereby may result in higher throughput to obtain rectified vapor 99. The rectified vapor 99 having temperature between 80-90 degree, pressure between 1.10-1.20 bar, and flowrate of 71710 kg/h present at the top of the rectifier column 15 may be further transferred to the first evaporator unit 30 for condensation process. The rectified vapor 99 may be condensed to form rectified condensate 48 in the first evaporator unit 30.

The part of rectified condensate 48 from the first evaporator unit 30 may be transferred back to the rectifier column 15 as a reflux 17 and the remaining rectified condensate 48 may be transferred to the dehydration section 89 for further concentration of ethanol from the rectified condensate 48. After condensation process the first evaporator unit 30 may comprise of water vapor 49 at temperature between 65-75-degree, pressure 0.2-0.4 Bar, and flowrate of 27520 kg/h. The water vapor 49 present at the top of the first evaporator unit 30 may be transferred to the Fan Set-II 24. Further the DDGS dryer unit 25 may transfer a dryer water vapor 60 to the scrubbing unit 10. The scrubbing unit 10 may be configured to purify the dryer water vapor 60 to obtain purified water vapor 46. The Fan Set-II 24 may be configured to receive the water vapor 49 from the first evaporator unit 30 and purified water vapor 46 from the scrubbing unit 10. The water vapor 49 may be at 0.2-0.4 bar pressure, flow rate of 27520 kg/h, and temperature of 65-75 degree and purified water vapor 46 may be at 0.2-0.4 bar pressure, flow rate of 13750 kg/h, and temperature of 70-80 degree may get compressed by the Fan Set-II 24 in one or more stages to obtain superheated water vapor 42 at pressure 0.7-0.9 bar and flow rate of 41270 kg/h.

The superheated water vapor 42 may be received by the first de-superheating unit 03. The first de-superheating unit 03 may be configured to de-superheat the superheated water vapor 42 to obtain saturated water vapor 53. Further, the saturated water vapor 53 may be received by the splitter unit 05. The splitter unit 05 may be configured to split the saturated water vapor 53 into saturated first water vapor stream 21 and saturated second water vapor stream 08. Further the saturated second water vapor stream 08 at the pressure between 0.7-0.9 bar, temperature 120-130 degree, and flow rate of 20100 kg/h may be transferred to the second evaporator unit 12 by the splitter unit 05. Further, the second evaporator unit 12 may be configured to receive an additional saturated water vapor stream 90 from the dehydration section 89. The additionally received saturated water vapor stream 90 from the dehydration section 89 and saturated second water vapor stream 08 received from splitter unit 05 may combine to form combined saturated water vapor 43. The second evaporator unit 12 may be configured to transfer the combined saturated water vapor 43 at a pressure of 0.4-0.6 bar, temperature of 80-90 degree, and flow rate of 24300 kg/h back to the analyser column 11 to make combined saturated water vapor 43 may act as a heat source.

Further the splitter unit 05 may be configured to transfer the saturated first water vapor stream 21 at a pressure of 0.7-0.9 bar, temperature of 90-100 degree, and flow rate of 24716 kg/h to the fan set-III 29. The fan set-III 29 may be configured to compress the saturated first water vapor stream 21 to a pressure of 2.1 bar to form superheated water vapor 07 at flow rate of 26516 kg/h. Further, the superheated water vapor 07 may be transferred to second second de-superheating unit 80. The second second de-superheating unit 80 may be configured to de-superheat the superheated water vapor 07 to obtain saturated water vapor 06 at pressure of 2.1 bar, temperature of 127° C. and flowrate of 26516 kg/h, a saturated water vapor 41 for the liquefaction section 90 at flowrate of 3671 kg/h, and a saturated water vapor 24 for the dehydration section 89. The saturated water vapor 06 at flow rate of 6800 kg/h may be received by the exhaust column 14, the saturated water vapor 41 may be received by the liquefaction section 90, and the saturated water vapor 24 may be received by the dehydration section 89 to act as a heat source.

The implementation of the above schematics may reduce steam consumption in the liquefication and dehydration section (not shown in the figure) due to additional water vapor fed from Fan Set III. Wherein, the additional water vapor may be generated by the association of DDGS Dryer with the current system. Further reduction of steam consumption in the liquefication and dehydration section may be configured to balance the optimization of process energy requirements, energy cost, and process carbon intensity.

The foregoing description shall be interpreted as illustrative and not in any limiting sense. A person of ordinary skill in the art would understand that certain modifications could come within the scope of this disclosure. The embodiments, examples and alternatives of the preceding paragraphs or the description and drawings, including any of their various aspects or respective individual features, may be taken independently or in any combination. Features described in connection with one embodiment are applicable to all embodiments unless such features are incompatible.

The improved system and method for ethanol distillation of the present subject matter has, but is not limited to, the following benefits/advantages:
No steam required.
useful for ethanol concentration and moisture removal.
pressure booster units compress intermediate stream (Mash Column top vapors) instead of using biofuel steam.
Increased pressure of rectifier column leads to additional distillation capacity and leading to modernization of ethanol plant capacity.
reduction of steam consumption in the liquefication and dehydration section due to additional water vapors generated in the Fan Set Il unit and Fan Set Ill unit reduces the dependency of the system on additional fuel-generated steam from outside sources.
optimizes process energy requirements, energy cost, and process carbon intensity

### List of Reference Numerals

19 Fermented wash

13 Preheater

20 Preheated feed stream

15′ Stripped vapor

79 Fan set-I

16 Compressed vapor

15 Rectifier column

99 Rectified vapor

30 First evaporating unit

17 Reflux

49 Water vapor

25 DDGS dryer unit

60 Dryer water vapor

10 Scrubbing unit

46 Purified water vapor

24 Fan set-II

42 Superheated water vapor

03 First de-superheating unit

53 Saturated water vapor

05 Splitter unit

21 First water vapor stream

08 Second water vapor stream

12 Second evaporating unit

43 Combined saturated water vapor

29 Fan set-III

07 Superheated water vapor

80 Second de-superheating unit

06 Saturated water vapor

14 Exhaust column

41 saturated water vapor for the liquefaction section

90 Liquefaction section

24 saturated water vapor for the dehydration section

89 Dehydration section

1-23. (canceled)

24. A method for distillation, the method comprising:
transferring a preheated feed stream to an analyser column configured to form a stripped vapor stream;
compressing the stripped vapor stream in a first fan unit sufficient to form compressed vapor;
transferring the compressed vapor to a rectifier column;
rectifying the compressed vapor in the rectifier column sufficient to form rectified vapor;
transferring the rectified vapor to a first evaporator unit;
condensing one or more vapor components of the rectified vapor in the first evaporator unit sufficient to form rectified condensate, wherein a water vapor stream is formed during the condensation process in the first evaporator unit; and
transferring the rectified condensate to the rectifier column and to a dehydration section.

25. The method of claim 24, wherein the compressed vapor entering the rectifier column increases operating pressure and temperature of the rectifier column sufficient to increase throughput of the rectifier column.

26. The method of claim 24 further comprising transferring the water vapor stream to a second fan unit, and compressing water vapor in the water vapor stream sufficient to form a first superheated water vapor stream.

27. The method of claim 26 further comprising de-superheating the first superheated water vapor stream in a first de-superheating unit, wherein the first de-superheating unit is configured to de-superheat the first superheated water vapor stream to form a first saturated water vapor stream.

28. The method of claim 27 further comprising transferring the first saturated water vapor stream to a splitter unit, wherein the splitter unit is configured to split the first saturated water vapor stream into a second saturated water vapor stream and a third saturated water vapor stream.

29. The method of claim 28 further comprising transferring the third saturated water vapor stream to a second evaporator unit, wherein the second evaporator unit is fluidically connected with the analyser column; and optionally transferring an additional saturated water vapor stream from the dehydration section to the second evaporator unit.

30. The method of claim 28 further comprising transferring the second saturated water vapor stream to a third fan unit, wherein the third fan unit is configured to compress water vapor in the second saturated water vapor stream sufficient to form a second superheated water vapor stream.

31. The method of claim 30 further comprising transferring the second superheated water vapor stream to a second de-superheating unit, wherein the second de-superheating unit is configured to de-superheat the second superheated water vapor stream sufficient to form a fourth saturated water vapor stream.

32. The method of claim 31 further comprising transferring the fourth saturated water vapor stream to an exhaust column, wherein the exhaust column is fluidically connected to the rectifier column.

33. The method of claim 31 further comprising transferring the fourth saturated water vapor stream to at least one of a liquefaction section and the dehydration section.

34. The method of claim 26 further comprising transferring a dryer water vapor from a DDGS dryer unit to a scrubbing unit, wherein the scrubbing unit is configured to purify the dryer water vapor to form purified water vapor, and wherein the purified water vapor is transferred to the second fan unit.

35. A system for distillation, the system comprising:
an analyser column configured for receiving a preheated feed stream and forming a stripped vapor stream;
a first fan unit configured to compress the stripped vapor stream in one or more stages sufficient to form compressed vapor;
a rectifier column configured to receive the compressed vapor sufficient to form rectified vapor; and
a first evaporator unit configured to condense one or more vapor components of the rectified vapor to form rectified condensate, wherein the rectified condensate is transferred to the rectifier column and a dehydration section, and wherein a water vapor stream is formed during the condensation process in the first evaporator unit.

36. The system of claim 35 further comprising a second fan unit configured to compress water vapor in the water vapor stream sufficient to form a first superheated water vapor stream.

37. The system of claim 36 further comprising a first de-superheating unit configured to de-superheat the first superheated water vapor stream sufficient to form a first saturated water vapor stream.

38. The system of claim 37 further comprising a splitter unit configured to split the first saturated water vapor stream into a second saturated water vapor stream and a third saturated water vapor stream.

39. The system of claim 38 further comprising a second evaporator unit configured for receiving the third saturated water vapor stream from the splitter unit, and optionally an additional saturated water vapor stream from the dehydration section, wherein the second evaporator unit is fluidically connected to the analyser column.

40. The system of claim 38 further comprising a third fan unit configured to compress water vapor in the second saturated water vapor stream sufficient to form a second superheated water vapor stream.

41. The system of claim 40 further comprising a second de-superheating unit configured to de-superheat the second superheated water vapor stream sufficient to form a fourth saturated water vapor stream.

42. The system of claim 41 further comprising an exhaust column, wherein the exhaust column is configured to receive the fourth saturated water vapor stream.

43. The system of claim 41 further comprising a liquefaction section, wherein one or more of the liquefaction section and the dehydration section are configured to receive the fourth saturated water vapor stream.

44. The system of claim 36 further comprising a DDGS dryer unit.

45. A system for distillation, the system comprising:
an analyser column configured for receiving a feed stream and forming a stripped vapor stream;
a first fan unit configured to compress the stripped vapor stream in one or more stages sufficient to form compressed vapor;
a rectifier column configured to rectify the compressed vapor sufficient to form rectified vapor;
a first evaporator unit configured to condense one or more vapor components of the rectified vapor to form rectified condensate, wherein the rectified condensate is transferred to the rectifier column and a dehydration section, and wherein a water vapor stream is formed during the condensation process in the first evaporator unit; and
a second fan unit configured to compress the water vapor in one or more stages.

46. The system of claim 45, wherein the first fan unit and the second fan unit each include one or more compressor stages, wherein the feed stream includes a preheated feed stream, and wherein the preheated feed stream includes a fermented wash at a temperature ranging from 60° C. to 70° C.
